(19) United States
(12) Patent Application Publication
(10) Pub. No.: US 2025/0069686 A1
(43) Pub. Date: Feb. 27, 2025

(54) METHODS AND SYSTEMS FOR PREDICTING PHENOTYPE

(71) Applicant: PIONEER HI-BRED INTERNATIONAL, INC., JOHNSTON, IA (US)

(72) Inventors: ERIC SCHULTZ, CLIVE, IA (US); ROLAND WELLE, STEINENSTADT (DE)

(73) Assignee: PIONEER HI-BRED INTERNATIONAL, INC., JOHNSTON, IA (US)

(21) Appl. No.: 18/724,926

(22) PCT Filed: Dec. 22, 2022
(86) PCT No.: PCT/US2022/082248

§ 371 Date: Jun. 27, 2024

Related U.S. Application Data
(60) Provisional application No. 63/266,064, filed on Dec. 28, 2021.

(51) Int. Cl. G16B 20/00; G06N 20/00; G16B 40/20

(52) U.S. Cl. CPC G16B 20/00; G06N 20/00; G16B 40/20

(57) ABSTRACT

Provided herein are systems and methods for characterizing one or more additional data profiles in plants or groups of plants. In one embodiment, a supervised learning model is established using one or more data profiles for two or more training plants or groups of training plants. Each plant or group of plants has a different identified phenotype for a phenotype of interest. Further, systems and methods for using the established supervised learning model to accurately predict a phenotype for a phenotype of interest for one or more test plants are also provided.

### CROSS-REFERENCE TO RELATED APPLICATIONS

This patent application claims the benefit of U.S. Provisional Application No. 63/266,064 filed on Dec. 28, 2021, which is incorporated herein by reference in its entirety.

### FIELD OF THE INVENTION

This disclosure relates generally to the fields of prediction, phenotypic analysis, and breeding.

### BACKGROUND OF THE INVENTION

Over the last 60 to 70 years, the contribution of plant breeding to agricultural productivity has been spectacular (Smith (1998) 53rd Annual corn and sorghum research conference, American Seed Trade Association, Washington, D.C.; Duvick (1992) Maydica 37:69). This has happened in large part because plant breeders have been adept at assimilating and integrating information from extensive evaluations of segregating progeny derived from multiple crosses of elite, inbred lines. Conducting such breeding programs requires extensive resources. A commercial maize breeder, for example, may evaluate 1,000 to 10,000 F3 topcrossed progeny derived from 100 to 200 crosses in replicated field trials across wide geographic regions. Therefore, plant breeders are interested in developing high yielding varieties and agronomically sound hybrids using less resources. Further, plant breeders are challenged with continually increasing the performance of their products to help meet the growing demand and future needs for food and feed supplies.

### SUMMARY

Provided herein are methods for predicting a phenotype of interest for at least one plant. The methods may include obtaining one or more data profiles from at least two groups of training plants, where the at least two groups of training plants have different identified phenotypes for a phenotype of interest, where the at least two groups of training plants are grown under the same conditions. The methods may also include using the one or more data profiles to establish a supervised learning model for predicting a phenotype of interest. The methods may also include predicting the phenotype of interest for at least one test plant by inputting a data profile from a test plant into the established supervised learning model to predict a phenotype for the phenotype of interest for the at least one test plant. Provided herein are methods for establishing a supervised learning model using the data profile of at least two groups of training plants. The methods may include characterizing the data profiles of at least two groups of training plants, where the at least two groups of training plants have different identified phenotypes for a phenotype of interest, where the at least two groups of training plants are grown under the same conditions. The methods may also include establishing a supervised learning model using as input the data profiles from the at least two groups of training plants, whereby the model analyzes or learns a phenotype for the phenotype of interest based on the data profiles. Provided herein are systems for use in predicting a phenotype of interest for a plant. The systems may include one or more servers, each of the one or more server storing plant data profiles and a computing device communicatively coupled to the one or more servers. The computing device may include a memory and one or more processors. In some aspects, the processors may be configured to obtain data profiles for two groups of training plants, where the data profiles from the two groups of plants have different identified phenotypes for a phenotype of interest, where the data profiles are obtained from the at least two groups of plants grown under the same conditions. In some aspects, the processors may be configured to analyze or learn phenotype prediction from the data profiles using a supervised learning model. In some aspects, the processors may be configured to obtain a data profile for a test plant. In some aspects, the processors may be configured to predict the phenotype of a phenotype of interest for the test plant.

### BRIEF DESCRIPTION OF THE SEVERAL VIEWS OF THE DRAWINGS

FIG. 1 shows the separation of drought (DRT) susceptible (diamond) versus DRT tolerant (square) hybrids after cross validated PLS-DA analysis. X axis refers to the samples (hybrids) and the Y-axis (YCV1 Predicted 1) describes the separation between the two classes—drought (DRT) susceptible versus DRT tolerant. Discrim Y1 is the optimal separation line for the assignment of these two classes.

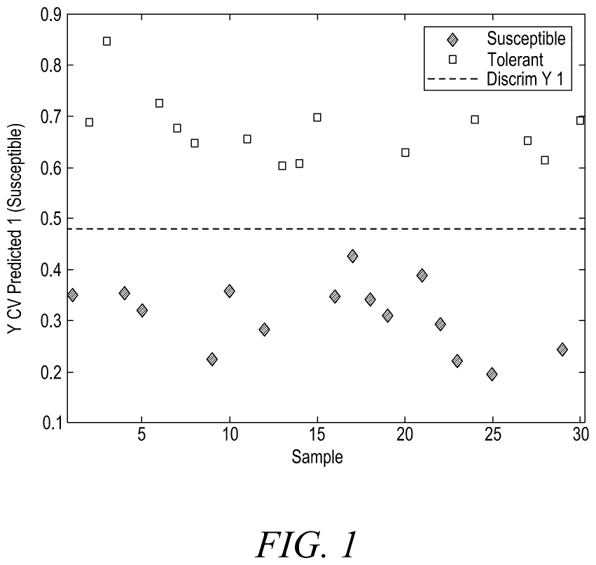

FIG. 2 is a flow chart of the individual analysis steps involved where the independent predictor variables e.g. metabolites, hyperspectral reflectance values, or SNPs are denoted X1 to Xn and the dependent DRT variable Y is coded by 0=Susceptible and 1=Tolerant or vice versa. The predictor variables are regressed on the categorical Y=DRT variable by e.g. an PLS-DA algorithm. Receiver Operator Curve (ROC) analysis using the predicted values from the PLS-DA modelling determines the ratio (AUC) between True Positives (prediction matches the reference value) and False Positives (prediction doesn't match the reference value). This ratio is also known as the AUC value (Area under the Curve) of an ROC analysis. The higher the AUC, the more predictions are accurate. A value of 1.0 means all predictions are accurate and a value of 0.5 means that half of the predictions are accurate and the other half is inaccurate, hence no information gain.

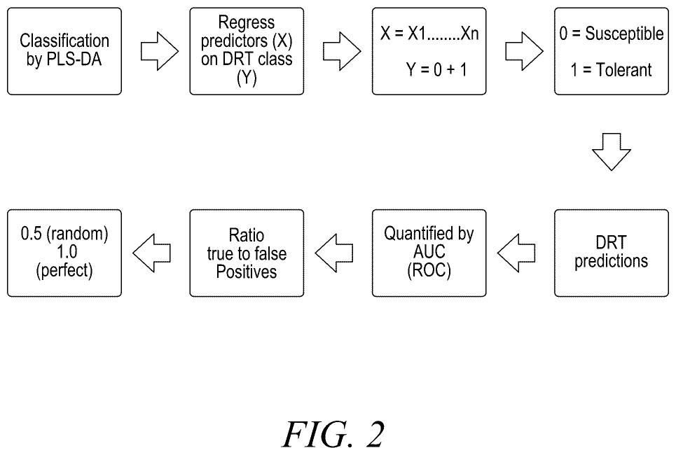

FIG. 3 is a schematic showing one embodiment for predicting drought tolerant hybrids using predictor variables, e.g. metabolites or SNPs. Hybrids with known DRT (drought) tolerant phenotypes and hybrids with drought-susceptible phenotypes are grown together with hybrids with unknown drought phenotypes under well-watered conditions or non-DRT (non-drought) stress conditions. Using hybrids with known DRT resistance/tolerant properties, a supervised learning model is built using e.g. PLS-DA analysis. In one embodiment, this PLS-DA model is then applied on the hybrids with unknown drought phenotypes (test hybrid plants) to predict the hybrids to be either tolerant or susceptible for DRT. The higher the prediction value the more drought tolerant the respective hybrid would be. The lower the prediction value the more drought susceptible the respective hybrid would be. Sorting the hybrids in descending order would show the most tolerant hybrids on top of the list.

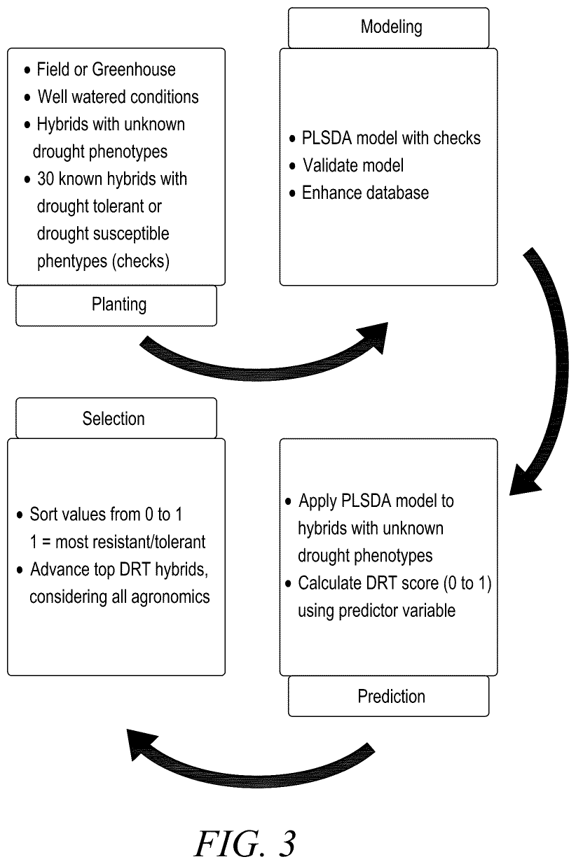

FIG. 4 shows the outcome in one embodiment of a supervised learning classification model. If the model has no classification power, the prediction accuracy is 50% (left side), meaning that no separation has taken place. If separation between the two classes is perfect, the prediction accuracy is 100% (right side).

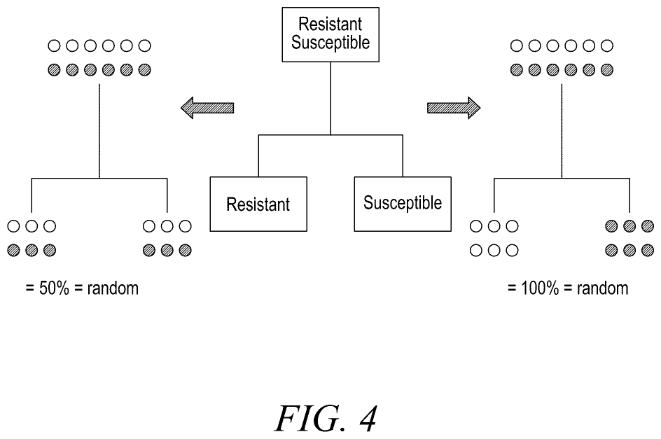

FIG. 5 is a block diagram illustrating an exemplary computer system including a server and a computing device according to one embodiment as disclosed herein.

### DETAILED DESCRIPTION OF THE INVENTION

It is to be understood that this invention is not limited to particular embodiments described herein, which can, of course, vary. Further, all publications referred to herein are each incorporated by reference for the purpose cited to the same extent as if each was specifically and individually indicated to be incorporated by reference herein.

Applying the phenotype prediction methods and systems disclosed herein to a breeding program allows a breeder to predict a plant's phenotype. The breeders may then decide which plants should be advanced and which plants should be culled from a breeding program, thereby providing savings in time, finances, laboratory and field resources, and labor.

Provided herein are systems and methods for characterizing one or more additional data profiles in training plants or groups of training plants.

In one embodiment, a supervised learning model is established using one or more data profiles for two or more training plants or groups of training plants. Each training plant or group of training plants has a different identified phenotype for a phenotype of interest, e.g. an agronomic trait of interest.

Further, systems and methods for using the established supervised learning model to accurately predict a phenotype for the phenotype of interest, e.g. an agronomic trait of interest, for one or more test plants are also provided.

The two or more training plants or groups of training plants used to establish the supervised learning model have different known or identified phenotypes for a phenotype of interest, e.g. an agronomic trait of interest, for example, one group of training plants may be drought tolerant while the other group of training plants may be drought susceptible. In some examples, the training plants or groups of plants are check plants. As used herein, a check plant, set, or group of check plants refers to a plant, set, or group of plants that have known trait properties, such as binary trait properties. The supervised learning model may be established using two or more groups of training plants as well as two or more individual training plants. The phenotypes for the two or more groups of plants may be well known, retrieved from a database, for example, from stored phenotypic profile information for the plants or populations, observed or obtained from the plants using any number of techniques, suitable methods, or sources, including automated instruments or systems.

In some aspects, the methods include growing the two or more training plants or groups of training plants with different known phenotypes for a phenotype of interest, e.g. an agronomic trait of interest, under the same conditions. The conditions may be non-stress or stress conditions. Exemplary non-stress conditions may include natural or man-made conditions including, but not limited to, adequate temperature, adequate pesticide treatment, appropriate soil type, adequate soil moisture, adequate nitrogen level, adequate herbicide treatment, appropriate day length, appropriate planting density, adequate light intensity, adequate light quality, appropriate tillage practice, appropriate day of planting, adequate carbon dioxide levels, adequate oxygen levels, nutrient sufficiency, low or no insect pressure, low or no disease pressure, low or no pathogens (e.g., bacteria, fungi, nematodes, viruses etc.), organisms (e.g., insects) as well as the absence or minimum amount of heavy metals, so that the plant is not stressed by its growing or environmental conditions. In some aspects, the plants subjected to the non-stress conditions are able to develop and grow without adverse impact as compared to a control plant subjected to stress growing or environmental conditions, such as poor environmental conditions, e.g. too little water. For example, same non-stress conditions may include growing the two groups of training plants under the same controlled light, temperature, water, nutrients and the like that foster normal plant growth and development. In some examples, normal plant growth and development means that the plant does not have stunted growth, and/or restricted development or altered metabolism as would occur in response to a plant stress condition or conditions.

In some examples, the two or more training plants or groups of training plants with different known or identified phenotypes for a phenotype of interest, such as drought tolerant plants and drought susceptible plants, are grown under the same non-stress conditions, for example, well-watered conditions or non-drought conditions. In some examples, the two groups of training plants may be grown in the same environment under the same conditions, e.g. grown in the same plot or field or individual pots in a greenhouse, so long as the stress and environmental conditions are the same.

In some aspects, the methods include growing the two or more training plants or groups of training plants with different known or identified phenotypes for a phenotype of interest, e.g. an agronomic trait of interest, under the same stress conditions. Exemplary stress conditions may include natural or man-made conditions including, but not limited to, inadequate temperature, inadequate pesticide treatment, inappropriate soil type, inadequate soil moisture, inadequate nitrogen level, inadequate herbicide treatment, inappropriate day length, inappropriate planting density, inadequate light intensity, inadequate light quality, inappropriate tillage practice, inappropriate day of planting, inadequate carbon dioxide levels, inadequate oxygen levels, nutrient insufficiency, high insect pressure, high disease pressure, high pathogens (e.g., bacteria, fungi, nematodes, viruses etc.), organisms (e.g., insects) as well as excess amount of heavy metals, so that the plant is stressed by its growing or environmental conditions. For example, same stress conditions may include growing the two groups of training plants under the same controlled light, temperature, water, nutrients and the like that alter a control plant's morphological, physiological, biochemical and/or molecular attributes or growth and development, as compared to the same control plant not subjected to the stress growing or environmental conditions, e.g. non-stressed conditions. In some examples, the control plant grown in the stress conditions may experience altered metabolism, stunted growth, and/or restricted development.

In some examples, the two or more training plants or groups of training plants with different known or identified phenotypes for a phenotype of interest, such as drought tolerant and drought susceptible plants, are grown under the same stress conditions, for example, watered-limited conditions. In some examples, the two groups of training plants may be grown in the same environment under the same conditions, e.g. grown in the same plot or field or individual pots in a greenhouse, so long as the stress and environmental conditions are the same.

In some aspects, one or more data profiles are obtained from the two or more plants or groups of training plants having differing phenotypes for the phenotype of interest and grown under the same conditions, e.g. stress or non-stress conditions. The data profiles may be stored in a database for ease of access and retrievability for use in establishing a supervised learning model.

The one or more data profiles may be genomic data profiles, transcriptomic data profiles, proteomic data profiles, metabolomic data profiles, spectral data profiles, or phenotypic data profiles, or combinations thereof. The data profile may include data from the whole plant. For example, the data profile may include genomic-wide plant data, transcriptomic-wide plant data, proteomic-wide plant data, metabolomic-wide plant data, hyperspectral-wide plant data, or phenotypic wide plant data. In some examples, the data may include component plant data or data from certain plant tissues or parts, such as leaves. For example, with respect to hyperspectral imaging, the component plant data may include data from the lower, middle, or upper parts of the plant.

The data may be obtained from any monocot or dicot plants, including but not limited to soybean, maize, sorghum, cotton, canola, sunflower, rice, wheat, sugarcane, alfalfa tobacco, barley, cassava, peanuts, millet, oil palm, potatoes, rye, or sugar beet plants. Accordingly, any monocot or dicot plants may be used with the methods, computing devices, computer-readable mediums and systems provided herein, including but not limited to a soybean, maize, sorghum, cotton, canola, sunflower, rice, wheat, sugarcane, alfalfa, tobacco, barley, cassava, peanuts, millet, oil palm, potatoes, rye, or sugar beet plant. In some examples, the plants are inbred or hybrid plants.

The data profiles for the two groups of training plants may be obtained from any suitable plants or parts thereof, for example, seeds, leaves, immature plants, or seedlings, such as V4-V10 growth stages. In some aspects, the data profiles are obtained from plants or parts thereof from a field, a greenhouse, or laboratory.

The data profiles may be obtained from the at least two groups of training plants or data set, one or more test plant or data set, or both. The test plants may have unknown or unidentified phenotypes for the phenotype of interest or unknown or unidentified phenotype classification for a phenotype of interest.

The data profiles may be based on information from one or more databases, one or more plants or group of plants, one or more cells, tissues, from a plant or groups of plants, one or more samples thereof, or any combination thereof. The data may be obtained using any suitable instrument, technique, or process. For example, phenotypic and trait data on morphological changes may be collected by visual observation. Such phenotypic and trait data may include, but is not limited to, characteristics such as normal plants, bushy plants, taller plants, thicker stalks, narrow leaves, striped leaves, knotted phenotype, chlorosis, albino, anthocyanin production, or altered tassels, ears, or roots. Other altered phenotypes and traits may be identified by measurements taken under field conditions, such as days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic and trait characteristics of harvested grain may be evaluated, including number of kernels per row on the ear, number of rows of kernels on the ear, kernel abortion, kernel weight, kernel size, kernel density and physical grain quality.

In particular embodiments of the invention, visual observation of plant phenotypes and traits may be collected using an automated system. In one such embodiment, the method of visual observation involves growing plants in a controlled greenhouse environment, transferring plants at selected times to an imaging analysis area where a quantitative, non-destructive light spectrum digital imaging analyzer, preferably having an instrumental variance below about 5%, takes reflected light images of the plant. The analyzer then analyzes those images to determine a value for a phenotypic or trait parameter of interest for the plant. Such an automated system is described in U.S. patent application Ser. No. 11/669,377, U.S. publication no. 20070186313, which is hereby incorporated by reference in its entirety.

A “genomic data profile” generally refers to a set of information about the entire genome of a given plant or group of plants (genome-wide), or it can encompass a specific subset of genome of a given plant or group of plants, or any combination thereof in a given plant or group of plants. In some aspects, the genomic data profile includes information regarding the presence or absence in the genome of a specific set of mutations, single nucleotide polymorphisms (SNPs), insertion of bases, deletion of bases, genotypic markers, other sequence information, or any combination thereof. In some examples, the data for genomic data profiles of the plants or groups of plants may be obtained using high density DNA arrays, PCR-based methods, including tape arrays, TaqMan assays, Restriction Fragment Length Polymorphisms (RFLPs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), or any combinations thereof.

A “phenotypic data profile” generally refers to the measurable characteristics of a plant that relate to a particular plant function or any combination thereof in a given plant or group of plants, for example, a set of information about the phenotypes, traits, or any combination thereof in a given plant or group of plants. In some examples, the data for phenotypic profiles of the plants or groups of plants may be obtained using any suitable technique or instrument for collecting one or more observable molecular, physical, or morphological characteristics of the plants.

In some embodiments, the phenotypic data profile includes but is not limited to data on root lodging resistance, stalk lodging resistance, brittle snap resistance, disease resistance, pest resistance, drought tolerance, cold tolerance, heat tolerance, salt tolerance, stress tolerance, herbicide tolerance, brittle stalk resistance, standability, metal tolerance, flowering time, color, fungal resistance, virus resistance, male sterility, female sterility, stalk strength, starch content, oil profile, amino acids balance, lysine level, methionine level, digestibility, fiber quality, plant growth, total plant area, transgene effects, response to chemical treatment, stress tolerance, gas exchange parameters, days to silk, days to shed, germination rate, biomass, dry shoot weight, nitrogen utilization efficiency, water use efficiency, nitrogen fixation, relative maturity, lodging, stress emergence rate, leaf senescence rate, canopy photosynthesis rate, silk emergence rate, anthesis to silking interval, percent recurrent parent, leaf angle, canopy width, leaf width ear fill, scattergrain, root mass, stalk strength, seed moisture, greensnap, shattering, visual pigment accumulation, kernels per ear, ears per plant, kernel density, leaf nitrogen content, grain nitrogen content, yield, including yield gain, grain yield, silage yield, yield drag, herbivore resistance, pathogen resistance, yield improvement, health enhancement, vigor, growth, ear size, grain moisture, photosynthetic capability improvement, plant height, pod number, pod position on the plant, number of internodes, number of kernels, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits, efficiency of germination, growth rate, ear relative maturity, ear number, seed number per ear, seed size, composition of seed (starch, oil, protein) and characteristics of seed fill, nutrition enhancement, altered protein content, altered oil content, increased biomass, increased shoot length, increased root length, improved root architecture, seed weight, altered seed carbohydrate composition, altered seed oil composition, altered seed protein composition, altered seed nutrient composition, or combinations thereof.

A “transcriptomic data profile” generally refers to a set of information about all the genes expressed in a given plant or group of plants (genome-wide transcriptomic), or it can encompass a specific subset of genes expressed in a given plant or group of plants or any combination thereof. In some aspects, the level of expression of the genes, temporal expression, spatial expression, or any combination thereof may be included in the transcriptomic profile. In some aspects, the transcriptomic data profile includes but is not limited to RNA transcript sequences and gene expression data by RNA sequence analysis. In some examples, the data for transcriptomic data profiles of the plants or groups of plants may be obtained using Microarray technology, Serial Analysis of Gene Expression (SAGE), RNA sequencing (RNA-Seq) and Massively Parallel Signature Sequencing (MPSS) or any other suitable technique.

A “proteomic data profile” generally refers to a set of information about all the proteins expressed by a given genome, given cell, given tissue or a given plant or group of plants at a certain time or it can encompass a specific subset of proteins expressed by a given genome, given cell, given tissue or a given plant or group of plants at a certain time or any combination thereof. In some aspects, the proteomic data profile includes but is not limited to protein sequences and protein expression data. In some examples, the data for proteomic data profiles of the plants or groups of plants may be obtained using SELDI, MALDI-MS, 2-D gel, or differential gel electrophoresis (DIGE) or any other suitable technique.

A “metabolomic data profile” generally refers a set of information about the metabolites of a given plant or group of plants. The term “metabolite” generally refers to a compound that is produced within an organism due to any process of anabolism or catabolism. In some aspects, the metabolomic data profile includes a set of metabolites, for example, low molecular weight compounds. For example, the data for metabolomic data profiles of the plants or groups of plants may be obtained using chromatography, mass spectrometers, nuclear magnetic resonance imaging (NMR) devices, and electrochemical arrays (EC). For example, the metabolomic data profile of a plant may be obtained by extracting the metabolites from a sample obtained from a plant, or cell, or plant part and detecting such metabolites by various analytical methods. The mass-to-charge fragments detected by mass spectrometry, may require several preprocessing steps including but not limited to signal denoising, alignment, baseline correction resulting in a metabolomic profile.

In some embodiments, additional normalization or standardization steps includes mathematical adjustments that allow comparison of values within and across datasets in a way that eliminates the effects of certain influences (e.g., error and anomalies), thereby decreasing experimental noise. In some examples, the data from the plants, including check plants, are normalized using one or more of the following criteria: across repetitions of an experiment, temperature differences, soil differences, location differences, biological replication, planting time differences, and any other desired condition or variable, or any combination thereof. In some examples, the data is normalized using a control plant or set of control plants. A control plant or set of control plants generally serves as a comparative reference point for normalization, for example, of plant handling logistics or environmental conditions in a greenhouse. For example, a control plant may comprise, a wild-type plant or a plant which is genetically similar to the training, validation, or test plants, where the plant is exposed to the same protocol or process as the training, validation, or test plant. Normalization may be performed by a suitable method described herein or known in the art.

In some aspects, the metabolomic data profiles comprise the entire set of metabolites for a whole plant which are detected and pre-processed.

A “spectral data profile” generally refers to a set of information of the electromagnetic spectrum for a three dimensional spatial area for a given plant or group of plants. In some aspects, the spectral data profile includes but is not limited hyperspectral or multi-spectral data. In some aspects, the hyperspectral data profile includes but is not limited to Red Green Blue (RGB) imaging data, infrared imaging data, ultra violet imaging data, wide spectrum imaging data, or any combinations thereof. Exemplary infrared imaging data includes but is not limited to near-infrared (NIR), far infrared (FIR), or thermal infrared imaging data and information or any combinations thereof. Exemplary spectral imaging includes but is not limited to hyperspectral or multispectral imaging data or information or any combinations thereof. In some aspects, the hyperspectral data profile includes but is not limited to absorbed or reflected wavelengths or bands of light, such as hyperspectral reflectance values, morphological data for a given plant within a group of plants, such as leaf area, number of leaves, leaf angle, perimeter of leaves, and plant height. For example, spectral data includes spectra from one or more wavelengths from the visible light spectrum, from the infrared spectrum, the near-infrared spectrum, the ultraviolet spectrum, the deep-ultraviolet spectrum, or any combination thereof. For example, the spectral data may include spectra of plants where all the spectra are taken at a wavelength of about 300 nm. In another example, the data may include spectra taken at wavelengths of from about 200 nm up to about 800 nm.

The spectral data may be from every wavelength along a range (e.g., at every wavelength from 200 nm up to about 2500 nm), or may be at intervals (e.g., at every other wavelength from 200 nm up to about 2500 nm, or even at every fifth wavelength from about 200 nm up to about 2500 nm). In some examples, the data for hyperspectral data profiles of the plants or groups of plants may be obtained using any suitable technique or instrument, such as a spectrometer, such as spectroradiometers, visible near infrared diffuse reflectance (VisNIR) spectroradiometer, and thermography, spectroscopy, or imaging systems, such as hyperspectral or multispectral cameras.

The various data profiles described herein may also include metadata, including but not limited to the individual plant's planting location, type of plant, and plant identification.

Described herein are methods and systems for predicting a phenotype for a phenotype of interest for a test plant that includes using an established supervised learning model. The supervised learning model may be established by using as input data profiles from two or more training plants or groups of plants that have different known phenotypes for a phenotype of interest. In some examples, the phenotype of interest is an agronomic trait of interest. In some aspects, the supervised learning model is established using data profiles obtained from two or more training plants or groups of training plants grown under the same conditions, where the two groups have different known phenotypes for a phenotype of interest, e.g. an agronomic trait of interest. In some aspects, one or more test plants are grown under the same conditions, e.g. stress or non-stress conditions, as the two or more training plants or groups of training plants. In some aspects, one or more test plants are grown together with the two or more training plants or two or more groups of training plants in the same plot, field, or greenhouse under the same conditions. In some aspects, one or more test plants are grown under the same conditions, e.g. stress or non-stress conditions, as the two or more training plants or groups of training plants and data is obtained from the training plants, test plants, or both for one or more data profiles, e.g. genomic data profiles, transcriptomic data profiles, proteomic data profiles, metabolomic data profiles, spectral data profiles, or phenotypic data profiles.

In some examples, the type of data profile for the two or more training plants or groups of training plants and the one or more test plants may be the same type of data profiles. In an example, if the input data for training the supervised learning model is the metabolomic data profile from two or more training plants or groups of plants, the input data for the established supervised learning model for a test plant may also be a metabolomic data profile.

In some embodiments, the supervised learning models may use any appropriate model that does not a priori know the relationship between the known phenotypes and the one or more data profiles of the two or more training plant groups prior to statistical analysis. In some embodiments, the supervised learning model is a classification model. In some embodiments, the supervised learning model is a regression model.

In some aspects, the supervised learning model uses multivariate analysis of the data profiles, e.g. from datasets of the two or more training plants, to relate the one or more data profiles to phenotypic data profiles. “Multivariate analysis” is intended to mean the use of any one of a number of statistical analyses, which are known to those of ordinary skill in the art, for analyzing data which arises from more than one variable. For example, the following formula may be used for determining multi-linear regression analysis of relationships between the set of independent variables and a single dependent variable to generate a supervised learning model that describes these relationships.

Y=b0+b1*x1+b2*x2+bn*xn, where Y is the dependent variable, b0 is where the line crosses the vertical access, b1-bn are the regression coefficients, X1-Xn are the independent variables which are multiplied by their respective regression coefficients.

Such techniques would allow for the establishment of a supervised learning model using the methods described herein.

In some embodiments, the multivariate analysis or analyses used to establish supervised learning models may include, but is not limited to, partial least squares analysis (PLS), partial least squares discriminant analysis (PLS-DA), latent variable techniques, cross-validation techniques, support vector machines, or neural networks, such as SVM-DA (Support Vector machines) or ANN-DA (Artificial Neural Networks), or deep learning algorithms, or combinations thereof.

In some embodiments, the supervised learning model is an inverse model.

Embodiments of the invention also encompass the identification and exclusion of outliers in the data sets. As used herein, “outliers” means the infrequent observations or data points which do not appear to follow the characteristic distribution of the rest of the data. As such, outliers may greatly influence the slope of the regression line and the value of the correlation coefficient. Such outliers may be identified and excluded by statistical methods including, but not limited to, cross-validation and principal component analysis. By “principal component analysis” is meant a statistical analysis that transforms a number of possibly correlated variables into a smaller number of uncorrelated variables called principal components, wherein the first principal component accounts for as much of the variability in the data as possible, and each succeeding component accounts for as much of the remaining variability as possible. The process of identifying outliers using cross-validation or principal component analysis is within the ability of one of ordinary skill in the art.

Further embodiments of the methods and systems include establishing a supervised learning model using support vector machines or neural networks and the data profiles as input. As used herein, “support vector machines” describe statistical analyses that are linear-classifier algorithms which determine a boundary (i.e., an n-dimensional hyperplane) which distinguishes between class members. The term “neural network” is intended to mean an actual or simulated (e.g., by computer program) network comprised of numerous, independent, highly interconnected artificial neurons which simulate the functions of biological neurons.

In some aspects, statistical analysis or analyses is used to determine the value of each variable (i.e., the data points of the data profile and phenotypic data profiles) in fitting the PLS or PLS-DA model for both predictors and response to the non-stress conditions. Such analysis or analyses may be applied to any of the methods or systems described herein for establishing the supervised learning model.

In some aspects, pre-processing steps are used to reduce the noise and dimensionality of the data prior to establishing the supervised learning models. In some aspects, the pre-processing steps reduce the noise and dimensionality of large data sets. As used herein, “pre-processing” of the data sets means to apply statistical analyses to the raw data in order to reduce the noise and dimensionality of the data. The term “dimensionality” refers to the number of variables under consideration in a data set. The term “noise” refers to the presence of any signal in the data set other than the signals which are desired for analysis. One or more statistical analyses or mathematical pretreatments may be used to pre-process the data in order to reduce the noise and dimensionality, including but not limited to multiplicative scatter correction, autoscaling, derivatization, or combinations thereof.

In some embodiments, the methods and systems include establishing a supervised learning model between the at least two training plants or groups of training plants that can be applied to a data profile of a test plant to predict a phenotype for a phenotype of interest, e.g. a phenotype for an agronomic trait of interest, in the test plant. In some aspects, the data profile of the test plant is used as input into the established supervised learning model.

In some examples, methods for predicting the at least one phenotype for phenotype of interest for at least one test plant includes inputting the data profile from a test plant into the established supervised learning model. The established supervised learning model may be used to predict at least one phenotype for a phenotype of interest for one or more plants based on the underlying relationships to the data, for example, tolerance or susceptibility to drought. In some aspects, the supervised learning model is used to predict at least one phenotype of interest using the data profiles from the two or more training plants or groups of plants grown under the same conditions. In some examples, the two or more training plants or groups of plants were grown under the same non-stress conditions. In some examples, the two or more training plants or groups of training plants were grown under the same stress conditions.

In some aspects, the test plant is not used to establish the supervised learning model. The test plant may be a plant at any stage of development, including an immature plant. Furthermore, the data profile of the test plant may be obtained at the same age or developmental stage as that of the training plants or groups of plants used to establish the supervised learning model or at a different stage as that of the plants used to establish the model.

In some examples, the methods include selecting one or more test plants based on its predicted phenotype for the phenotype of interest. The methods may include selecting one or more test plants having a desired predicted value or classification for the phenotype of interest, e.g. agronomic trait of interest. In some examples, the established supervised learning model provides a predicted value for the test plant and assigns a classification to the predicted value, for example, a drought classification, such as tolerant or susceptible to drought. In some examples, the one or more selected plants are predicted to exhibit an improved or increased desirable phenotype or agronomic trait of interest, such increased yield, increased drought resistance, improved brittle stalk resistance, or improved standability.

The systems and methods herein may provide for predicting a phenotype or an agronomic trait of interest for a plant, which includes, but is not limited to, the following: disease resistance, drought tolerance, standability, yield, brittle snap, heat tolerance, cold tolerance, salinity tolerance, metal tolerance, herbicide tolerance, root lodging resistance, stalk lodging resistance, improved water use efficiency, nitrogen utilization, nitrogen fixation, pest resistance, herbivore resistance, pathogen resistance, yield improvement, health enhancement, vigor, growth, ear size, grain moisture, photosynthetic capability improvement, plant height, pod number, pod position on the plant, number of internodes, number of kernels, incidence of pod shatter, grain size, efficiency of nodulation and nitrogen fixation, efficiency of nutrient assimilation, resistance to biotic and abiotic stress, carbon assimilation, plant architecture, percent seed germination, seedling vigor, juvenile traits, efficiency of germination, growth rate, ear relative maturity, ear number, seed number per ear, seed size, composition of seed (starch, oil, protein) nutrition enhancement, biomass, shoot length, root length, root architecture, seed weight, altered seed carbohydrate composition, altered seed oil composition, altered seed protein composition, or altered seed nutrient composition, flowering time, color, fungal resistance, virus resistance, stalk strength, amino acids balance, lysine level, methionine level, digestibility, fiber quality, plant growth, total plant area, transgene effects, response to chemical treatment, stress tolerance, gas exchange parameters, days to silk, days to shed, germination rate, dry shoot weight, relative maturity, stress emergence rate, leaf senescence rate, canopy photosynthesis rate, silk emergence rate, anthesis to silking interval, leaf angle, canopy width, leaf width ear fill, scattergrain, root mass, stalk strength, seed moisture, greensnap, shattering, kernels per ear, ears per plant, kernel density, leaf nitrogen content, grain nitrogen content, yield, including yield gain, grain yield, silage yield, yield drag, herbivore resistance, pathogen resistance, yield improvement, ear size, grain moisture, photosynthetic capability improvement, plant height, pod position on the plant, number of internodes, number of kernels, resistance to lodging, or combinations thereof.

In some examples, the methods include selecting one or more test plants based on its predicted phenotype of interest or phenotype classification. The methods may include selecting one or more test plants based on its AUC value. The higher the AUC value, the more likely the predicted phenotype of interest or phenotype classification is accurate and true. An AUC value of 1.0 represents the highest possible accuracy, as all predictions are true. In some examples, the test plants may be selected if the AUC value is 1.0 or greater than 0.5, 0.6, 0.7, 0.8, or 0.9. In some examples, the established supervised learning model provides a predicted value for the test plant and assigns a classification to the predicted value for the phenotype, for example, a drought classification, such as tolerant to drought or susceptible to drought, or a yield classification, such as increased yield or decreased yield, or a standability classification, such as improved standability or decreased standability. In some examples, the AUC value is used to assign to the one or more test plants a predicted phenotype or phenotype classification, for example, a drought classification or phenotype such as tolerant to drought or susceptible to drought.

In some examples, the one or more selected plants are predicted to exhibit a phenotype of an improved or increased desirable phenotype of interest, such increased yield, increased drought resistance/tolerance, or improved standability. Other examples of phenotypes of interest are described elsewhere herein. In some examples, the methods include growing one or more of the selected plants having a desired predicted phenotype or phenotype classification in a plant growing environment, such as a greenhouse, a laboratory, a field, or any other suitable environment. These one or more selected plants, when grown, are expected exhibit an improved or increased desirable phenotype of interest, such as increased yield, increased drought resistance, or improved standability, as compared to a check plant or a set of check plants. As used herein, a check plant or set of check plants refers to a plant or set of plants that have known trait properties, such as binary trait properties. In some embodiments, the check plant or set of check plants may be used to establish the supervised learning model and/or validate the results from the trained/established supervised learning model. These selected plants may be included or advanced in a breeding program and/or further evaluated. Accordingly, the methods may also include growing the selected plants or a part thereof in a plant growing environment, such as a greenhouse, a laboratory, a field, or any other suitable environment. The one or more selected test plants that are predicted to have at least one desirable phenotype of interest or desired phenotype classification may be crossed with another plant. The selected test plant or plant derived therefrom may be bred with at least one other plant or selfed, e.g., to create a new line or hybrid, used in recurrent selection, bulk selection, or mass selection, backcrossed, used in pedigree breeding or open pollination breeding, and/or used in genetic marker enhanced selection. In some instances, a plant having at least one predicted desirable phenotype of interest may be crossed with another plant or back-crossed so that a desirable genotype may be introgressed into the plant by sexual outcrossing or other conventional breeding methods. In some examples, selected plants having at least one predicted desirable phenotype of interest may be used in crosses with another plant from to generate a population of progeny. The plants may be grown and crossed according to any breeding protocol relevant to the particular breeding program. The one or more selected plants, progeny from crosses, or parts thereof may be used in a breeding program.

In some examples, the methods include selecting one or more plants, based on the predicted phenotype of interest or phenotype classification. The methods may include selecting one or more members of the test population or test plants having an undesired predicted value for the phenotype of interest. In some examples, the one or more selected plants exhibit an unimproved or less improved, poorer, or undesirable phenotype of interest or phenotype classification, such decreased yield, increased drought susceptibility, or decreased standability, compared to a check plant or a set of check plants. Plants predicted to have at least one undesirable or less improved phenotype of interest, e.g. poorer yield, may be counter-selected and removed from a breeding program.

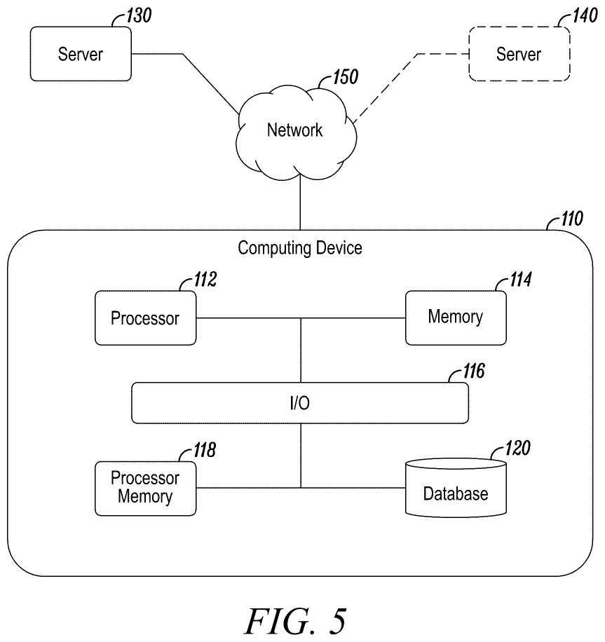

Referring to FIG. 5, a block diagram of a computer system 100 for predicting a phenotype of interest, e.g. an agronomic trait of interest, is shown. To do so, the system 100 may include a computing device 110 and a server 130 that is associated with a computer system. The system 100 may further include one or more servers 140 that are associated with other computer systems such that the computing device 110 may communicate with different computer systems running different platforms. However, it should be appreciated that, in some embodiments, a single server (e.g., a server 130) may run multiple platforms. The computing device 110 is communicatively coupled to the one or more servers 130, 140 via a network 150 (e.g., a local area network (LAN), a wide area network (WAN), a personal area network (PAN), the Internet, etc.).

In another embodiment, the computing device 110 may generate predictions for one or more phenotypes of interest by using at least one supervised learning model or any other suitable model that has been trained or established using data profiles from two groups of training plants grown under the same conditions. The two groups of training plants have different known or identified phenotypes for a phenotype of interest, for example, one group of training plants may be known to be drought tolerant, and the other group of training plants may be known to be drought susceptible.

The data profiles may be genomic data profiles, transcriptomic data profiles, proteomic data profiles, metabolomic data profiles, spectral data profiles, or phenotypic data profiles. Accordingly, the one or more training datasets may include genomic data profiles, transcriptomic data profiles, proteomic data profiles, metabolomic data profiles, spectral data profiles, and/or phenotypic data profiles.

More specifically, the computing device 110 may obtain data profiles stored in a database 120 and/or input or downloaded by a user. For example, in the context of predicting a plant's phenotype for a phenotype of interest a supervised learning model may be used to predict a phenotype for a test plant using data profiles from two groups of training plants that have different identified phenotypes for a phenotype of interest, e.g. a trait of interest, and grown under the same conditions.

In general, the computing device 110 may include any existing or future devices capable of training or using a supervised learning model or any other suitable model. For example, the computing device may be, but not limited to, a computer, a notebook, a laptop, a mobile device, a smartphone, a tablet, wearable, smart glasses, or any other suitable computing device that is capable of communicating with the server 130.

The computing device 110 includes a processor 112, a memory 114, an input/output (I/O) controller 116 (e.g., a network transceiver), a memory unit 118, and a database 120, all of which may be interconnected via one or more address/data bus. It should be appreciated that although only one processor 112 is shown, the computing device 110 may include multiple processors. Although the I/O controller 116 is shown as a single block, it should be appreciated that the I/O controller 116 may include a number of different types of I/O components (e.g., a display, a user interface (e.g., a display screen, a touchscreen, a keyboard), a speaker, and a microphone).

The processor 112 as disclosed herein may be any electronic device that is capable of processing data, for example a central processing unit (CPU), a graphics processing unit (GPU), a system on a chip (SoC), or any other suitable type of processor. It should be appreciated that the various operations of example methods described herein (i.e., performed by the computing device 110) may be performed by one or more processors 112. The memory 114 may be a random-access memory (RAM), read-only memory (ROM), a flash memory, or any other suitable type of memory that enables storage of data such as instruction codes that the processor 112 needs to access in order to implement any method as disclosed herein. It should be appreciated that, in some embodiments, the computing device 110 may be a computing device or a plurality of computing devices with distributed processing.

As used herein, the term “database” may refer to a single database or other structured data storage, or to a collection of two or more different databases or structured data storage components. In the illustrative embodiment, the database 120 is part of the computing device 110. In some embodiments, the computing device 110 may access the database 120 via a network such as network 150. The database 120 may store data (e.g., input, output, intermediary data) used for predicting a phenotype of interest. For example, the data may include data profiles that are obtained from one or more servers 130, 140.

The computing device 110 may further include a number of software applications stored in a memory unit 118, which may be called a program memory. The various software applications on the computing device 110 may include specific programs, routines, or scripts for performing processing functions associated with the methods described herein. Additionally or alternatively, the various software applications on the computing device 110 may include general-purpose software applications for data processing, database management, data analysis, network communication, web server operation, or other functions described herein or typically performed by a server. The various software applications may be executed on the same computer processor or on different computer processors. Additionally, or alternatively, the software applications may interact with various hardware modules that may be installed within or connected to the computing device 110. Such modules may implement part of or all of the various exemplary method functions discussed herein or other related embodiments.

Although only one computing device 110 is shown in FIG. 5, the server 130, 140 is capable of communicating with multiple computing devices similar to the computing device 110. Although not shown in FIG. 5, similar to the computing device 110, the server 130, 140 also includes a processor (e.g., a microprocessor, a microcontroller), a memory, and an input/output (I/O) controller (e.g., a network transceiver). The server 130, 140 may be a single server or a plurality of servers with distributed processing. The server 130, 140 may receive data from and/or transmit data to the computing device 110.

The network 150 is any suitable type of computer network that functionally couples at least one computing device 110 with the server 130, 140. The network 150 may include a proprietary network, a secure public internet, a virtual private network and/or one or more other types of networks, such as dedicated access lines, plain ordinary telephone lines, satellite links, cellular data networks, or combinations thereof. In embodiments where the network 150 comprises the Internet, data communications may take place over the network 150 via an Internet communication protocol.

As used herein, the term “plant” also includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

As used in this specification and the appended claims, terms in the singular and the singular forms “a,” “an,” and “the,” for example, include plural referents unless the content clearly dictates otherwise. Thus, for example, reference to “plant,” “the plant,” or “a plant” also includes a plurality of plants; also, depending on the context, use of the term “plant” can also include genetically similar or identical progeny of that plant.

The following embodiments are offered by way of illustration and not by way of limitation.

Embodiment 1. A method for predicting a phenotype of interest for at least one

plant, said method comprising:
obtaining one or more data profiles from at least two groups of training plants, wherein said at least two groups of training plants have different identified phenotypes for a phenotype of interest, wherein the at least two groups of training plants are grown under the same conditions;
using the one or more data profiles to establish a supervised learning model for predicting a phenotype of interest; and
predicting the phenotype of interest for at least one test plant by inputting a data profile from a test plant into the established supervised learning model to predict a phenotype for the phenotype of interest for the test plant.

Embodiment 2. A method for predicting a phenotype of interest for at least one plant, said method comprising:
a) growing at least two groups of training plants under the same conditions, wherein each group of training plants has different identified phenotypes of interest;
b) obtaining one or more data profiles from the at least two groups of training plants;
c) using the one or more data profiles to establish a supervised learning model; and
d) predicting the phenotype of interest for at least one test plant by inputting a data profile from a test plant into the established supervised learning model to predict the phenotype of interest for the test plant.

Embodiment 3. A method for predicting a phenotype of interest for at least one plant, said method comprising:
a) obtaining one or more genotypic data profiles from at least two groups of training plants, wherein each group of training plants has different identified phenotypes of interest;
b) using the genotypic data profile to establish a supervised learning model; and
c) predicting the phenotype of interest for at least one plant by inputting a genotypic data profile from a test plant into the established supervised learning model to predict the phenotype of interest for the test plant.

Embodiment 4. A method for predicting a phenotype of interest for at least one plant, said method comprising: predicting a phenotype of interest for at least one test plant by inputting a genotypic data profile from a test plant into an established supervised learning model to predict the phenotype of interest for the test plant.

Embodiment 5. A method for predicting a phenotype of interest for at least one plant, said method comprising: predicting a phenotype of interest for at least one test plant by inputting a data profile from a test plant into an established supervised learning model to predict the phenotype of interest for the test plant.

Embodiment 6. A method for establishing a supervised learning model using the data profile of at least two groups of training plants, said method comprising:

characterizing the data profiles of at least two groups of training plants, wherein said at least two groups of training plants have different identified phenotypes for a phenotype of interest, wherein the at least two groups of training plants are grown under the same conditions; and
establishing a supervised learning model using as input the data profiles from the at least two groups of training plants, whereby the model predicts a phenotype of interest for a test plant based on the data profiles.

Embodiment 7. The method of embodiment 5, the method comprising using data profiles from at least two groups of training plants as input to establish the supervised learning model, wherein each group of training plants has different identified phenotypes for a phenotype of interest, and wherein the data profiles were obtained from the at least two groups of training plants grown under the same conditions.

Embodiment 8. The method of embodiment 1, 3, 6, or 7, the method comprising growing the at least two groups of training plants under the same non-stress conditions.

Embodiment 9. The method of embodiment 1, 3, 6, or 7, growing the at least two groups of training plants under the same stress conditions.

Embodiment 10. The method of embodiment 1 or 2, wherein the one or more data profiles comprises genomic data profiles, transcriptomic data profiles, proteomic data profiles, metabolomic data profiles, spectral data profiles, or phenotypic data profiles.

Embodiment 11. The method of embodiment 5, wherein the data profile comprises a genomic data profile, transcriptomic data profile, proteomic data profile, metabolomic data profile, spectral data profile, or phenotypic data profile.

Embodiment 12. The method of embodiment 6 or 7, wherein the data profiles comprise genomic data profiles, transcriptomic data profiles, proteomic data profiles, metabolomic data profiles, spectral data profiles, or phenotypic data profiles.

Embodiment 13. The method of embodiment 1, 2, 3, 4, 5, or 6, wherein the supervised learning model is a regression or classification model.

Embodiment 14. The method of embodiment 1, 2, 3, 4, 5, or 6, wherein the supervised learning model is an inverse model.

Embodiment 15. The method of embodiment 6, further comprising predicting the phenotype of interest for at least one plant by inputting a data profile from a test plant into the established supervised learning model to predict the phenotype of interest for the test plant.

Embodiment 16. The method of embodiment of embodiment 1, 2, 3, 4, 5, or 15 further comprising selecting the at least one test plant based on the predicted phenotype of interest.

Embodiment 17. The method of embodiment 16, further comprising growing the selected at least one test plant in a plant growing environment.

Embodiment 18. The method of embodiment 1, 2, 3, 4, 5, or 6, wherein the phenotype of interest is an agronomic trait of interest.

Embodiment 19. The method of embodiment 1, 2, 3, 4, 5, or 6, wherein the phenotype of interest comprises disease resistance, drought tolerance, standability, yield, heat tolerance, cold tolerance, salinity tolerance, metal tolerance, herbicide tolerance, improved water use efficiency, nitrogen utilization, nitrogen fixation, pest resistance, herbivore resistance, or pathogen resistance.

Embodiment 20. The method of embodiment 3 or 4, the method further comprising: using one or more genotypic data profiles to establish the supervised learning model, wherein genotypic data profiles are from at least two groups of training plants, wherein each group of training plants has different identified phenotypes for a phenotype of interest.

Embodiment 21. The method of embodiment 1 or 2, wherein the test plant is grown under the same conditions, e.g. same stress or same non-stress conditions, as the two or more training plants or two or more groups of training plants and the data for the test plant obtained therefrom is used in the one or more data profiles, e.g. genomic data profiles, transcriptomic data profiles, proteomic data profiles, metabolomic data profiles, spectral data profiles, or phenotypic data profiles.

Embodiment 22. The method of embodiment 6, wherein the test plant is grown under the same conditions, e.g. same stress or same non-stress conditions, as the two or more training plants or two or more groups of training plants and the data for the test plant obtained therefrom is used in the data profiles, e.g. genomic data profiles, transcriptomic data profiles, proteomic data profiles, metabolomic data profiles, spectral data profiles, or phenotypic data profiles.

Embodiment 23. The method of embodiment 7, wherein the test plant is grown under the same conditions, e.g. same stress or same non-stress conditions, as the two or more training plants or two or more groups of training plants and the data for the test plant obtained therefrom is used in the data profile, e.g. a genomic data profile, a transcriptomic data profile, a proteomic data profile, a metabolomic data profile, a spectral data profile, or a phenotypic data profile.

Embodiment 24. The method of embodiment 3, wherein the test plant is grown under the same conditions, e.g. same stress or same non-stress conditions, as the two or more training plants or two or more groups of training plants and the data for the test plant obtained therefrom is used in one or more genomic data profiles for the test plant.

Embodiment 25. The method of embodiment 20, wherein the test plant is grown under the same conditions, e.g. same stress or same non-stress conditions, as the two or more training plants or two or more groups of training plants and the data for the test plant obtained therefrom is used in a genomic data profile for the test plant.

Embodiment 26. The method of any of the embodiments of embodiment 1, 2, 3, 7, 15, or 20, wherein the data profile of the test plant and the data profiles of the training group of plants are the same type of data profiles.

Embodiment 27. The method of any of the embodiments of embodiment 1, 2, 3, 4, 5, or 6, wherein the supervised learning model is established using multivariate analysis or analyses of one or more of the data profiles, e.g. PLS or PLS-DA analysis, to relate the one or more data profiles to one or more phenotypic data profiles.

Embodiment 28. The method of any of the embodiments of embodiment 1, 2, 3, 4, 5, 6, or 27, wherein the supervised learning model's performance for phenotype prediction is evaluated using ROC (Receiver Operating Characteristics) analysis.

Embodiment 29. The method of any of the embodiments of embodiment 1, 2, 3, 4, 5, 6, or 28, wherein the probability of the accuracy of the predicted phenotype is evaluated using AUC (Area Under The Curve) values.

Embodiment 30. A method for classifying at least one plant for a phenotype of interest, said method comprising:

obtaining one or more data profiles from at least two groups of training plants, wherein said at least two groups of training plants have different identified phenotypes for a phenotype of interest, wherein the at least two groups of training plants are grown under the same conditions;
using the one or more data profiles to establish a supervised learning model for classifying a plant for a phenotype of interest; and
classifying at least one test plant for a phenotype of interest by inputting a data profile from the one test plant into the established supervised learning model to classify the test plant for the phenotype of interest.

Embodiment 31. A method for classifying at least one plant for a phenotype of interest, said method comprising:
e) growing at least two groups of training plants under the same conditions, wherein each group of training plants has different identified phenotypes of interest;
f) obtaining one or more data profiles from the at least two groups of training plants;
g) using the one or more data profiles to establish a supervised learning model; and
h) classifying at least one test plant for a phenotype of interest by inputting a data profile from the one test plant into the established supervised learning model to classify the test plant for the phenotype of interest.

Embodiment 32. A method for classifying at least one plant for a phenotype of interest, said method comprising:
d) obtaining one or more genotypic data profiles from at least two groups of training plants, wherein each group of training plants has different identified phenotypes of interest;
e) using the genotypic data profile to establish a supervised learning model; and
f) classifying the at least one test plant for a phenotype of interest by inputting a genotypic data profile from the one test plant into the established supervised learning model to classify the test plant for the phenotype of interest.

Embodiment 33. A method for classifying at least one plant for a phenotype of interest, said method comprising: classifying the at least one test plant for a phenotype of interest by inputting a genotypic data profile from the test plant into an established supervised learning model to classify the test plant for the phenotype of interest.

Embodiment 34. A method for classifying at least one plant for a phenotype of interest, said method comprising: classifying the at least one test plant for a phenotype of interest by inputting a data profile from a test plant into an established supervised learning model to classify the test plant for the phenotype of interest.

Embodiment 35. A method for establishing a supervised learning model using the data profile of at least two groups of training plants, said method comprising:

characterizing the data profiles of at least two groups of training plants, wherein said at least two groups of training plants have different identified phenotypes for a phenotype of interest, wherein the at least two groups of training plants are grown under the same conditions; and
establishing a supervised learning model using as input the data profiles from the at least two groups of training plants, whereby the model classifies a plant for a phenotype of interest based on the data profiles.

Embodiment 36. The method of embodiment 34, the method comprising using data profiles from at least two groups of training plants as input into the supervised learning model, wherein each group of training plants has different identified phenotypes for a phenotype of interest, and wherein the data profiles were obtained from the at least two groups of training plants grown under the same conditions.

Embodiment 37. The method of embodiment 30, 31, 35, or 36, the method comprising growing the at least two groups of training plants under the same non-stress conditions.

Embodiment 38. The method of embodiment 30, 32, 35, or 36, growing the at least two groups of training plants under the same stress conditions.

Embodiment 39. The method of embodiment 30 or 31, wherein the one or more data profiles comprises genomic data profiles, transcriptomic data profiles, proteomic data profiles, metabolomic data profiles, spectral data profiles, or phenotypic data profiles.

Embodiment 40. The method of embodiment 34, wherein the data profile comprises a genomic data profile, transcriptomic data profile, proteomic data profile, metabolomic data profile, spectral data profile, or phenotypic data profile.

Embodiment 41. The method of embodiment 35 or 36, wherein the data profiles comprise genomic data profiles, transcriptomic data profiles, proteomic data profiles, metabolomic data profiles, spectral data profiles, or phenotypic data profiles.

Embodiment 42. The method of embodiment 30, 31, 32, 33, 34, 35, or 36, wherein the supervised learning model is a regression or classification model.

Embodiment 43. The method of embodiment 30, 31, 32, 33, 34, 35, or 36, wherein the supervised learning model is an inverse model.

Embodiment 44. The method of embodiment 35, further comprising classifying at least one test plant for a phenotype of interest by inputting a data profile from the test plant into the established supervised learning model to generate a classification for a phenotype for the phenotype of interest for the test plant.

Embodiment 45. The method of embodiment of embodiment 30, 31, 32, 33, 34, or 44 further comprising selecting the at least one test plant based on the classification of the phenotype of interest.

Embodiment 46. The method of embodiment 45, further comprising growing the selected at least one test plant in a plant growing environment.

Embodiment 47. The method of embodiment 30, 31, 32, 33, 34, 35, or 36, wherein the phenotype of interest is an agronomic trait of interest.

Embodiment 48. The method of embodiment 30, 31, 32, 33, 34, 35, or 36, wherein the phenotype of interest comprises disease resistance, drought tolerance, standability, yield, heat tolerance, cold tolerance, salinity tolerance, metal tolerance, herbicide tolerance, improved water use efficiency, nitrogen utilization, nitrogen fixation, pest resistance, herbivore resistance, or pathogen resistance.

Embodiment 49. The method of embodiment 32 or 33, the method further comprising: using one or more genotypic data profiles to establish the supervised learning model, wherein genotypic data profiles are from at least two groups of training plants, wherein each group of training plants has different identified phenotypes for a phenotype of interest.

Embodiment 50. The method of embodiment 30 or 31, wherein the test plant is grown under the same conditions, e.g. same stress or same non-stress conditions, as the two or more training plants or two or more groups of training plants and the data for the test plant obtained therefrom is used in the one or more data profiles, e.g. genomic data profiles, transcriptomic data profiles, proteomic data profiles, metabolomic data profiles, spectral data profiles, or phenotypic data profiles.

Embodiment 51. The method of embodiment 35, wherein the test plant is grown under the same conditions, e.g. same stress or same non-stress conditions, as the two or more training plants or two or more groups of training plants and the data for the test plant obtained therefrom is used in the data profiles, e.g. genomic data profiles, transcriptomic data profiles, proteomic data profiles, metabolomic data profiles, spectral data profiles, or phenotypic data profiles.

Embodiment 52. The method of embodiment 36, wherein the test plant is grown under the same conditions, e.g. same stress or same non-stress conditions, as the two or more training plants or two or more groups of training plants and the data for the test plant obtained therefrom is used in the data profile, e.g. a genomic data profile, a transcriptomic data profile, a proteomic data profile, a metabolomic data profile, a spectral data profile, or a phenotypic data profile.

Embodiment 53. The method of embodiment 32, wherein the test plant is grown under the same conditions, e.g. same stress or same non-stress conditions, as the two or more training plants or two or more groups of training plants and the data for the test plant obtained therefrom is used in one or more genomic data profiles for the test plant.

Embodiment 54. The method of embodiment 49, wherein the test plant is grown under the same conditions, e.g. same stress or same non-stress conditions, as the two or more training plants or two or more groups of training plants and the data for the test plant obtained therefrom is used in a genomic data profile for the test plant.

Embodiment 55. The method of any of the embodiments of embodiment 30, 31, 32, 36, 44, or 49, wherein the data profile of the test plant and the data profiles of the training group of plants are the same type of data profiles.

Embodiment 56. The method of any of the embodiments of embodiment 30, 31, 32, 33, 34, 35, or 36, wherein the supervised learning model is established using multivariate analysis or analyses of one or more of the data profiles, e.g. PLS or PLS-DA analysis, to relate the one or more data profiles to one or more phenotypic data profiles.

Embodiment 57. The method of any of the embodiments of embodiment 30, 31, 32, 33, 34, 35, 36, or 56, wherein the supervised learning model's performance for phenotype classification is evaluated using ROC (Receiver Operating Characteristics) analysis.

Embodiment 58. The method of any of the embodiments of embodiment 30, 31, 32, 33, 34, 35, 36, or 57, wherein the probability of the accuracy of the phenotype classification is evaluated using AUC (Area Under The Curve) values.

Embodiment 59. A computing device comprising a processor configured to perform the steps in of any of the methods of embodiments 1-58.

Embodiment 60. A computer-readable medium comprising instructions which, when executed by a computing device, cause the computing device to carry out the steps in any of the methods of embodiments 1-58.

Embodiment 61. A system for use in predicting a phenotype of interest for one or more plants, the system comprising:
one or more servers, each of the one or more server storing plant data profiles; and
a computing device communicatively coupled to the one or more servers, the computing device including:
   a memory; and
   one or more processors configured to:
      obtain data profiles for two groups of training data plants, wherein the data profiles from the two groups of plants have different identified phenotypes for a phenotype of interest, wherein the data profiles are obtained from the at least two groups of plants grown under the same conditions;
      analyze or learn phenotype prediction from the data profiles using a supervised learning model;
      obtain at least one data profile for one or more test plants; and
      predict a phenotype of the phenotype of interest for the one or more test plants.

Embodiment 62. A system for use in classifying one or more plants for a phenotype of interest, the system comprising:

one or more servers, each of the one or more server storing plant data profiles; and
a computing device communicatively coupled to the one or more servers, the computing device including:
a memory; and
one or more processors configured to:
   obtain data profiles for two groups of training data plants, wherein the data profiles from the two groups of plants have different identified phenotypes for a phenotype of interest, wherein the data profiles are obtained from the at least two groups of plants grown under the same conditions;
   analyze or learn phenotype classification from the data profiles using a supervised learning model;
   obtain at least one data profile for one or more test plants; and
   classify the one or more test plants for a phenotype classification for the phenotype of interest.

Embodiment 63. The system of embodiment 61 or 62, wherein the data profiles are obtained from the at least two groups of plants grown under the same non-stress conditions.

Embodiment 64. The system of embodiment 61 or 62, wherein the data profiles are obtained from the at least two groups of plants grown under the same stress conditions.

Embodiment 65. The system of embodiment 61 or 62, wherein the data profiles comprise genomic data profiles, transcriptomic data profiles, proteomic data profiles, metabolomic data profiles, spectral data profiles, or phenotypic data profiles.

Embodiment 66. The system of embodiment 61 or 62, wherein the data profile for the one or more test plant comprises a genomic data profile, transcriptomic data profile, proteomic data profile, metabolomic data profile, spectral data profile, or phenotypic data profile.

Embodiment 67. The system of embodiment 61 or 62, wherein the supervised learning model is a regression or classification model.

Embodiment 68. The system of embodiment 61 or 62, wherein the supervised learning model is an inverse model.

Embodiment 69. The system of embodiment 61 or 62, the system receiving as input the data profile from the one or more test plants for the established supervised learning model.

Embodiment 70. The system of embodiment 61, wherein the system comprises a processor configured to select the one or more test plants based on the one or more test plants predicted phenotype for the phenotype of interest.

Embodiment 71. The system of embodiment 62, wherein the system comprises a processor configured to select the one or more test plants based on the one or more test plant's phenotype classification for the phenotype of interest.

Embodiment 72. The system of embodiment 61 or 62, wherein the phenotype of interest is an agronomic trait of interest.

Embodiment 73. The system of embodiment 61 or 62, wherein the phenotype of interest comprises disease resistance, drought tolerance, standability, yield, heat tolerance, cold tolerance, salinity tolerance, metal tolerance, herbicide tolerance, improved water use efficiency, nitrogen utilization, nitrogen fixation, pest resistance, herbivore resistance, or pathogen resistance.

Embodiment 74. The system of embodiment 61 or 62, wherein the at least one test plant is grown under the same conditions, e.g. same stress conditions or same non-stress conditions, as the two or more training plants or two or more groups of training plants and the data for the at least one test plant obtained therefrom is used in the one or more data profiles, e.g. genomic data profiles, transcriptomic data profiles, proteomic data profiles, metabolomic data profiles, spectral data profiles, or phenotypic data profiles.

Embodiment 75. The system of embodiment 61 or 62, wherein the at least one data profiles for the at least one test plant comprises genomic data profiles, transcriptomic data profiles, proteomic data profiles, metabolomic data profiles, spectral data profiles, or phenotypic data profiles.

Embodiment 76. The system of embodiment 61 or 62, wherein the at least one data profile of the at least one test plant and the data profiles of the two or more training groups of plants are the same type of data profiles.

Embodiment 77. The system of embodiment 61 or 62, wherein the supervised learning model is established using multivariate analysis or analyses of one or more of the data profiles, e.g. PLS or PLS-DA analysis, to relate the one or more data profiles to one or more phenotypic data profiles.

Embodiment 78. The system of embodiment 61, wherein the probability of the accuracy of the predicted phenotype is evaluated using AUC (Area Under The Curve).

Embodiment 79. The system of embodiment 61, wherein the system further comprises a processor configured to calculate an AUC value for the probability of the accuracy of the predicted phenotype.

Embodiment 80. The system of embodiment 61 or 79, wherein the system comprises a processor configured to select the one or more test plants based on one or more AUC values.

Embodiment 81. The system of embodiment 62, wherein the probability of the accuracy of the phenotype classification is evaluated using AUC (Area Under The Curve).

Embodiment 82. The system of embodiment 62, wherein the system further comprises a processor configured to calculate an AUC value for the probability of the accuracy of the phenotype classification.

Embodiment 83. The system of embodiment 62 or 82, wherein the system comprises a processor configured to select the one or more test plants based on one or more AUC values.

### EXAMPLES

The following examples are offered by way of illustration and not by way of limitation.

### EXAMPLES

### Example 1—Identifying DRT Resistant Hybrids from Europe 2019 Under Well-Watered Conditions Using Field or Greenhouse Grown Plants Based on Metabolite Profiling

Corn hybrids were classified as being DRT (drought) tolerant or DRT (drought) susceptible under non-DRT stress conditions using the metabolome. Hybrids of known DRT class property were planted in Seville, Spain or in a greenhouse environment in Johnston, USA under well-watered conditions. Three diverse maturity groups were used, Europe Maritime (EUMA) with CRM 83-97, Europe Continental (EUCO) with CRM 100-107, and Europe Mediterranean (EUME) with CRM 107-113, which covers most European corn genetic diversity. Each maturity group consisted of 10 hybrids, 5 being known as DRT tolerant and 5 being known as DRT susceptible. In total there were 30 hybrids (15 DRT tolerant, 15 DRT susceptible), with CRM ranging from 83-113. The field replication factor was 40, with 4 plot reps and 10 plants per plot rep. The greenhouse replication factor was 20, with a total of 20 individual plants in a specific design. Sampling in the field was at the onset of flowering, while in the greenhouse sampling occurred at 3 different v-stages, i.e. v5, v7, and v10.

### Gas Chromatograph and Time of Flight Mass Spectrometer Settings and Methods

To characterize the metabolic profile of each plant or group of plants, metabolites were extracted from four lyophilized leaf discs of approximately 3 mg combined dry weight. Five hundred microliters (500 μL) of chloroform:methanol:water (2:5:2, v/v/v) containing 0.015 mg ribitol internal standard were added to each sample in a 1.1 mL polypropylene microtube containing two 5/32″ (3.97 mm) stainless steel ball bearings. Samples were homogenized in a 2000 Geno/Grinder ball mill at setting 1,650 for 1 minute and then rotated at 4° C. for 30 minutes. Samples were then centrifuged at 1,454 g for 15 minutes at 4° C. Next, 300 μL aliquots were transferred to 1.8 mL high recovery GC vials and subsequently evaporated to dryness in a speed vac. The dried residues were re-dissolved in 50 μL of 20 mg mL⁻¹ methoxyamine hydrochloride in pyridine, capped, and agitated with a vortex mixer. The samples were incubated in an orbital shaker at 30° C. for 90 minutes to form methoxyamine derivatives. Eighty microliters of N-methyl-N-(trimethylsilyl) trifluoroacetamide (MSTFA) were added to each sample to form trimethylsilyl derivatives. The MSTFA delivery to individual samples was performed by the gas chromatograph autosampler 30 minutes prior to injection, greatly minimizing sample variability due to differences in the state of derivatization.

Trimethylsilyl derivatives were separated by gas chromatography on a Restek 20 m×0.18 mm id×0.18 μm film thickness Rtx®-5Sil MS column. One microliter (1 μL) injections were made with a 1:30 split ratio using a Leco autosampler. The Agilent 7890B gas chromatograph was programmed for an initial temperature of 80° C. for 0.1 minutes, increased to 320° C. at 36° minute⁻¹ where it was held for 3.8 minutes before being cooled rapidly to 80° C. in preparation for the next run. The injector and transfer line temperatures were 270° C. and 300° C., respectively, and the source temperature was 250° C. Helium was used as the carrier gas with a constant flow rate of 0.8 mL min⁻¹ maintained by electronic pressure control. Data acquisition was performed on a LECO Pegasus BT time-of-flight mass spectrometer with an acquisition rate of 30 spectra s⁻¹ in the mass range of m/z 49-600. An electron beam of 70 eV was used to generate spectra. Detector voltage was approximately 1999-2051 V depending on the instrument and detector age. An instrument auto tune for mass calibration using perfluorotributylamine (PFTBA) was performed prior to each GC sequence.

### Pre-Processing of Raw GC/ToFMS Data

Raw Leco GC/MS.peg data files were converted into netcdf (Andi) formats using the Leco ChromaTof® software. For each netcdf file, retention times were converted into C10-C36-derived retention indices using an in-house program. Data preprocessing which consisted of chromatogram gridding in the m/z value and retention index dimensions, chemical noise subtraction, aligning the retention indices of each selected ion chromatogram, detecting nominal mass peaks, and peak grouping was performed with Genedata Refiner MS™ software. For the latter data reduction step, all peaks within the same retention index window (0.75 s) that corresponded to the same compound were coalesced into a one group based on normalized Euclidean distance measurements between the individual intensity profiles across all samples. Minimum group size was three peaks. The resulting data matrix consisted of intensities for each of the m/z value and retention index combinations (peak groups) for each sample. The aligned and de-noised data matrix was then transferred to Genedata Analyst™ software with which each intensity value was normalized for both ribitol internal standard signal and sample dry weight. The data were further corrected for batch (sequence) and run order effects using a mixed model scripted in R.

### Modeling and Results

In modeling, all data were pre-processed by autoscaling, or dividing each data channel by its standard deviation in the dataset, followed by mean centering. In each case, partial least squares-discriminant analysis (PLS-DA) was applied to build qualitative calibrations to classify hybrids based on the metabolites as predictors. The 2 DRT states were digitally represented as ones and zeros. Leave-one-hybrid-out cross-validation was used to select the number of latent variables and provide DRT predictions for the hybrids. This method removed all the reps of a given hybrid from the training set. A model was built on the remaining hybrids and used to predict the DRT status of all reps of the one hybrid left out of the modeling process. This process was repeated for all hybrids. All predictions for all hybrids were combined, resulting in a fully-predicted, cross-validated dataset. Outliers were identified using principal component analysis (PCA), but other unsupervised methods may also be used. All modeling was performed using the PLS Toolbox from Eigenvector Research. Predictions derived from the PLS-DA models were further analyzed by Receiver Operating Curves (ROC), where the AUC values (Area Under the Curve) provided a quantitative measure for the accuracy of the classification into DRT resistant versus DRT susceptible hybrids. AUC values range from 0.5-1.0, where 0.5 means a random assay as 50% of the predictions are true and the other 50% are false. The higher the AUC, the better the underlying assay can be used to predict DRT membership. Hence a value of 1.0 represents the highest possible accuracy, as all predictions are true. The datasets were analyzed either for each individual maturity group or the combination of all 3 groups in one database. All AUC values were at 1.0 for field metabolites. While greenhouse results were calculated for all individual v-stages, the results tables below show the AUC values for the average of all v-stages ranges from 0.71 to 0.86. The best v-stage result is always better than the average of all v-stages (see tables, below).

| | maturity groups | | | |
|---|---|---|---|---|
| | EUMA | EUCO | EUME | EUMA-CO-ME |
| predictor variables | field metabolites | | | |
| no of hybrids | 10 | 10 | 10 | 30 |
| AUC DRT | 1.00 | 1.00 | 1.00 | 1.00 |

| | maturity groups | | | |
|---|---|---|---|---|
| | EUMA | EUCO | EUME | EUMA-CO-ME |
| predictor variables | greenhouse metabolites, v-stage averages | | | |
| no of hybrids | 10 | 10 | 10 | 30 |
| AUC DRT | 0.71 | 0.99 | 0.92 | 0.86 |
| best v-stage | 0.80 | 1.00 | 0.95 | 0.90 |

### Example 2—Identifying DRT Resistant Hybrids from Europe 2019 Under Well-Watered Conditions Using Greenhouse Grown Plants Based on Hyperspectral Imaging

Corn hybrids were classified as being DRT tolerant or DRT susceptible under non-DRT stress conditions using hyperspectral imaging (HSI). Hybrids of known DRT class property were planted in a greenhouse environment in Johnston, USA under well-watered conditions. Three diverse maturity groups were used, EUMA (CRM 83-97), EUCO (100-107), and EUME (107-113), which covers most European corn genetic diversity. Each maturity group consisted of 10 hybrids, 5 being known as DRT tolerant and 5 being known as DRT susceptible. In total there were 30 hybrids (15 DRT tolerant, 15 DRT susceptible), with CRM ranging from 83-113. The greenhouse replication factor was 20, with a total of 20 individual plants in a specific design. Hyperspectral images were collected at v5, v6, v7, v9, and v10 stages.

### Hyperspectral Imaging Settings and Methods

Hyperspectral imaging (HSI) was used to collect visual and near-infrared phenotype. The cameras used collect reflectance spectra from 400-1000 nm wavelengths at 1.5 nm intervals. Each plant was individually imaged at regular intervals during the growth phase in line with growing conditions using hyperspectral imaging stations in an automated greenhouse environment. Imaging times aligned with v-stages v5, v6, v7, v9, and v10. For each timepoint, total spectra were preprocessed to remove background signals, leaving only plant tissue spectra. Using a different set of proprietary processes, physiologically relevant features, also called “HSI-Traits” like leaf area, leaf angle, number of leaves were calculated from the plant spectra (58 Traits in total). These HS-Traits and the individual plant spectra were normalized and standardized to remove instrument-to-instrument variation.

### Modeling and Results

In modeling, all data were pre-processed by autoscaling, or dividing each data channel by its standard deviation in the dataset, followed by mean centering. In each case, partial least squares-discriminant analysis (PLS-DA) was applied to build qualitative calibrations to classify hybrids based on the hyperspectral imaging data as predictors. The 2 DRT states were digitally represented as ones and zeros. Leave-one-hybrid-out cross-validation was used to select the number of latent variables and provide DRT predictions for the hybrids. This method removed all the reps of a given hybrid from the training set. A model was built on the remaining hybrids and used to predict the DRT status of all reps of the one hybrid left out of the modeling process. This process was repeated for all hybrids. All predictions for all hybrids were combined, resulting in a fully-predicted, cross-validated dataset. Outliers were identified using principal component analysis (PCA). All modeling was performed using the PLS Toolbox from Eigenvector Research. Predictions derived from the PLS-DA models were further analyzed by Receiver Operating Curves (ROC), where the AUC values (Area Under the Curve) provided a quantitative measure for the accuracy of the classification into DRT resistant versus DRT susceptible hybrids. AUC values range from 0.5-1.0, where 0.5 means a random assay as 50% of the predictions are true and the other 50% are false. The higher the AUC, the better the underlying assay can be used to predict DRT membership. Hence a value of 1.0 represents the highest possible accuracy, as all predictions are true. The datasets were analyzed either for each individual maturity group or the combination of all 3 groups in one database. While greenhouse results were calculated for all individual v-stages, the results table below shows the AUC values for the average of all v-stages, which ranges from 0.64 to 0.98. The best v-stage result for HSI-Traits is always better than the average of all v-stages (see table, below).

| | maturity groups | | | |
|---|---|---|---|---|
| | EUMA | EUCO | EUME | EUMA-CO-ME |
| predictor variables | greenhouse HSI-Traits, v-stage averages | | | |
| no of hybrids | 10 | 10 | 10 | 30 |
| AUC DRT | 0.88 | 0.84 | 0.98 | 0.64 |
| best v-stage | 0.92 | 0.96 | 1.00 | 0.68 |

### Example 3—Identifying DRT Resistant Hybrids from Europe 2019 Using SNP Profiling

Corn hybrids were classified as being DRT tolerant or DRT susceptible under non-DRT stress conditions using SNP profiling. Three diverse maturity groups were used, EUMA (CRM 83-97), EUCO (100-107), and EUME (107-113), which covers most European corn genetic diversity. Each maturity group consisted of 10 hybrids, 5 being known as DRT tolerant and 5 being known as DRT susceptible. In total there were 30 hybrids (15 DRT tolerant, 15 DRT susceptible), with crm ranging from 83-113.

### SNP Profiling Settings and Methods

SNP markers were collected from the homozygeous inbred parents of the hybrids. Briefly, DNA was sampled and assayed using the Infinium XT High Density microarray (Illumina, Inc.). From all scored SNPs, a subset of 2511 polymorphic markers were used. Missing scores were imputed using an in-house IBD Identity by Descent framework but could be imputed using any suitable technique or method. Allelic scores were transformed into binary numeric values (0/1). Hybrids were made in silico by combining the allelic scores of the female and male inbreds, which were then used for subsequent modeling efforts.

### Modeling and Results

In modeling, all data were pre-processed by autoscaling, or dividing each data channel by its standard deviation in the dataset, followed by mean centering. In each case, partial least squares-discriminant analysis (PLS-DA) was applied to build qualitative calibrations to classify hybrids based on the SNPs as predictors. The 2 DRT states were digitally represented as ones and zeros. Leave-one-hybrid-out cross-validation was used to select the number of latent variables and provide DRT predictions for the hybrids. This method removed all the reps of a given hybrid from the training set. A model was built on the remaining hybrids and used to predict the DRT status of all reps of the one hybrid left out of the modeling process. This process was repeated for all hybrids. All predictions for all hybrids were combined, resulting in a fully-predicted, cross-validated dataset. Outliers were identified using principal component analysis (PCA). All modeling was performed using the PLS Toolbox from Eigenvector Research. Predictions derived from the PLS-DA models were further analyzed by Receiver Operating Curves (ROC), where the AUC values (Area Under the Curve) provided a quantitative measure for the accuracy of the classification into DRT resistant versus DRT susceptible hybrids. AUC values range from 0.5-1.0, where 0.5 means a random assay as 50% of the predictions are true and the other 50% are false. The higher the AUC, the better the underlying assay can be used to predict DRT membership. Hence a value of 1.0 represents the highest possible accuracy, as all predictions are true. The datasets were analyzed either for each individual maturity group or the combination of all 3 groups in one database.

| | maturity groups | | | |
|---|---|---|---|---|
| | EUMA | EUCO | EUME | EUMA-CO-ME |
| predictor variables | SNPs | | | |
| no of hybrids | 10 | 10 | 10 | 30 |
| AUC DRT | 0.60 | 0.96 | 1.00 | 0.95 |

### Example 4—Identifying DRT Resistant Hybrids from Europe 2020 and US 2020 Using SNP Profiling

Corn hybrids were classified as being DRT tolerant or DRT susceptible under non-DRT stress conditions using SNP profiling. Two different regions were used for this analysis. For the Europe dataset, 78 R4 and R5 hybrids came from EUMA (CRM 83-97), EUCO (100-107), and EUME (107-113) maturity groups, which covers most European corn genetic diversity. Of this population, 52 hybrids were DRT tolerant and 26 hybrids were DRT susceptible. For the US dataset, 88 R5 hybrids came from early (crm 85-103) and late (crm 105-115) maturity groups, which covers most US R5 corn genetic diversity. Of this population, 25 hybrids were DRT tolerant and 63 hybrids were DRT susceptible. The combined dataset (EU+US) had 166 hybrids with 77 hybrids being DRT tolerant and 89 hybrids being DRT susceptible and crm ranging from 83 to 115.

### SNP Profiling Settings and Methods

SNP markers were collected from the homozygeous inbred parents of the hybrids. Briefly, DNA was sampled and assayed using the Infinium XT High Density microarray (Illumina, Inc.). From all scored SNPs, a subset of 2511 polymorphic markers were used. Missing scores were imputed using an in-house IBD framework but could be imputed using any suitable technique or method. Allelic scores were transformed into binary numeric values (0/1). Hybrids were made in silico by combining the allelic scores of the female and male inbreds, which were then used for subsequent modeling efforts.

### Modeling and Results

In modeling, all data were pre-processed by autoscaling, or dividing each data channel by its standard deviation in the dataset, followed by mean centering. In each case, partial least squares-discriminant analysis (PLS-DA) was applied to build qualitative calibrations to classify hybrids based on the SNPs as predictors. The 2 DRT states were digitally represented as ones and zeros. Leave-one-hybrid-out cross-validation was used to select the number of latent variables and provide DRT predictions for the hybrids. This method removed all the reps of a given hybrid from the training set. A model was built on the remaining hybrids and used to predict the DRT status of all reps of the one hybrid left out of the modeling process. This process was repeated for all hybrids. All predictions for all hybrids were combined, resulting in a fully-predicted, cross-validated dataset. Outliers were identified using principal component analysis (PCA). All modeling was performed using the PLS Toolbox from Eigenvector Research. Predictions derived from the PLS-DA models were further analyzed by Receiver Operating Curves (ROC), where the AUC values (Area Under the Curve) provided a quantitative measure for the accuracy of the classification into DRT resistant versus DRT susceptible hybrids. AUC values range from 0.5-1.0, where 0.5 means a random assay as 50% of the predictions are true and the other 50% are false. The higher the AUC, the better the underlying assay can be used to predict DRT membership. Hence a value of 1.0 represents the highest possible accuracy, as all predictions are true. The datasets were analyzed either individually or as a merged database between EU und US corn genetics. These datasets with R4 and R5 hybrids were less thoroughly characterized for DRT as compared to the mostly commercial hybrids from Examples 1-3 as they underwent only 1 or 2 years of DRT characterization. Since the input data for the Y property was less strong than Examples 1-3, it follows that results would not be as strong either. Therefore, we see lower AUC values than in previous examples, but the values in this example are still high enough for making appropriate selections for DRT resistant hybrids. Additionally, the merged dataset of EU+US hybrids—with a very strong diversity in maturity and corn genetics—still displayed a strong AUC of 0.83. An interesting dataset consisting of 13 hybrids containing the same transgene was also tested for DRT with an AUC of 1.00, i.e. all of the transgenic hybrids were well classified into their DRT resistant or susceptible groups.

| | maturity groups | | | |
|---|---|---|---|---|
| | EU early + | US early + | | US late with |
| | late | late | EU + US | transgene |
| predictor variables | SNPs | | | |
| no of hybrids | 78 | 88 | 166 | 138 |
| AUC DRT | 0.77 | 0.86 | 0.83 | 0.88 |

### Example 5—Identifying Brittle Resistant Hybrids from US 2020 Using SNP Profiling

Corn hybrids were classified as being brittle-tolerant or brittle-susceptible under non-DRT stress conditions using SNP profiling. Two different datasets were used for this analysis. The first dataset used 64 R5 hybrids from late maturity (crm 105-115), 34 of which were brittle-tolerant and the remaining 30 hybrids were brittle-susceptible. The second dataset was composed of 25 R5 hybrids from late maturity (crm 105-115) containing a transgene or not (Wildtype). In this set, 11 hybrids were brittle-tolerant and 14 hybrids were brittle-susceptible.

### SNP Profiling Settings and Methods

SNP markers were collected from the homozygeous inbred parents of the hybrids. Briefly, DNA was sampled and assayed using the Infinium XT High Density microarray (Illumina, Inc.). From all scored SNPs, a subset of 2511 polymorphic markers were used. Missing scores were imputed using an in-house IBD framework. Allelic scores were transformed into binary numeric values (0/1). Hybrids were made in silico by combining the allelic scores of the female and male inbreds, which were then used for subsequent modeling efforts.

### Modeling and Results

In modeling, all data were pre-processed by autoscaling, or dividing each data channel by its standard deviation in the dataset, followed by mean centering. In each case, partial least squares-discriminant analysis (PLS-DA) was applied to build qualitative calibrations to classify hybrids based on the SNPs as predictors. The 2 brittle states were digitally represented as ones and zeros. Leave-one-hybrid-out cross-validation was used to select the number of latent variables and provide brittle predictions for the hybrids. This method removed all the reps of a given hybrid from the training set. A model was built on the remaining hybrids and used to predict the brittle-status of all reps of the one hybrid left out of the modeling process. This process was repeated for all hybrids. All predictions for all hybrids were combined, resulting in a fully-predicted, cross-validated dataset. Outliers were identified using principal component analysis (PCA). All modeling was performed using the PLS Toolbox from Eigenvector Research. Predictions derived from the PLS-DA models were further analyzed by Receiver Operating Curves (ROC), where the AUC values (Area Under the Curve) provided a quantitative measure for the accuracy of the classification into brittle-resistant versus brittle-susceptible hybrids. AUC values range from 0.5-1.0, where 0.5 means a random assay as 50% of the predictions are true and the other 50% are false. The higher the AUC, the better the underlying assay can be used to predict brittle-membership. Hence a value of 1.0 represents the highest possible accuracy, as all predictions are true. The datasets were analyzed either individually or as a merged database between EU und US corn genetics. These datasets with R5 hybrids were less thoroughly characterized for brittle as compared to the mostly commercial brittle-resistant hybrids from Examples 1-3 as they underwent only 2 years of brittle characterization. Hence the input data for the Y property was not as strong due to less characterization and ground truthing. While the US late had an AUC of 0.77, the transgenic dataset displayed a very high AUC of 0.93. These results indicate that brittle can also be classified with PLS-DA using SNPs as predictors in a similar way than already proven for brittle-resistance.

maturity groups

| | |
|---|---|
| US late | US (with transgene) |

| | |
|---|---|
| predictor variables | SNPs |

| | | |
|---|---|---|
| no of hybrids | 64 | 25 |
| AUC Brittle | 0.77 | 0.93 |

1. A method for predicting a phenotype of interest for at least one plant, said method comprising:
obtaining one or more data profiles from at least two groups of training plants, wherein said at least two groups of training plants have different identified phenotypes for a phenotype of interest, wherein the at least two groups of training plants are grown under the same conditions;
using the one or more data profiles to establish a supervised learning model for predicting a phenotype of interest; and
predicting the phenotype of interest for at least one test plant by inputting a data profile from a test plant into the established supervised learning model to predict a phenotype for the phenotype of interest for the at least one test plant.

2. A method for establishing a supervised learning model using the data profile of at least two groups of training plants, said method comprising:
characterizing one or more data profiles of at least two groups of training plants, wherein said at least two groups of training plants have different identified phenotypes for a phenotype of interest, wherein the at least two groups of training plants are grown under the same conditions; and
establishing a supervised learning model using as input the one or more data profiles from the at least two groups of training plants, whereby the model predicts a phenotype for the phenotype of interest based on the one or more data profiles.

3. The method of claim 1 or 2, growing the at least two groups of training plants under the same non-stress conditions or same stress conditions.

4. The method of claim 1, wherein the one or more data profiles comprises genomic data profiles, transcriptomic data profiles, proteomic data profiles, metabolomic data profiles, spectral data profiles, or phenotypic data profiles.

5. The method of claim 1 or 2, wherein the supervised learning model is a regression or classification model.

6. The method of claim 1, further comprising selecting the at least one test plant based on the predicted phenotype for the phenotype of interest.

7. The method of claim 6, further comprising growing the selected at least one test plant in a plant growing environment.

8. The method of claim 1 or 2, wherein the phenotype of interest is an agronomic trait of interest.

9. The method of claim 1 or 2, wherein the phenotype of interest comprises disease resistance, drought tolerance, standability, yield, heat tolerance, cold tolerance, salinity tolerance, metal tolerance, herbicide tolerance, improved water use efficiency, nitrogen utilization, nitrogen fixation, pest resistance, or herbivore resistance.

10. The method of claim 2, wherein the data profiles comprise genomic data profiles, transcriptomic data profiles, proteomic data profiles, metabolomic data profiles, spectral data profiles, or phenotypic data profiles.

11. The method of claim 2, further comprising predicting a phenotype for the phenotype of interest for at least one plant by inputting a data profile from a test plant into the established supervised learning model to predict a phenotype for the phenotype of interest for the test plant.

12. The method of claim 11, the method comprising selecting the at least one test plant based on the predicted phenotype for the phenotype of interest.

13. The method of claim 12, the method comprising further comprising growing the selected at least one test plant in a plant growing environment.

14. The method of any of the claims of claim 1 or 12, wherein the data profile of the test plant and the data profiles of the at least two training groups of plants are the same type of data profiles.

15. The method of claim 1 or 2, wherein the supervised learning model is established using multivariate analysis of the one or more data profiles.

16. The method of claim 1, 2, or 15, wherein the supervised learning model is established using multivariate analysis of the one or more data profiles to relate the one or more data profiles to phenotypic data profiles.

17. The method of claim 16, wherein the supervised learning model's performance for phenotype prediction is evaluated using ROC (Receiver Operating Characteristics) analysis and AUC (Area Under The Curve) values.

18. A system for use in predicting a phenotype of interest for a plant, the system comprising:
one or more servers, each of the one or more server storing plant data profiles; and
a computing device communicatively coupled to the one or more servers, the computing device comprising:
a memory; and
one or more processors configured to:
obtain data profiles for two groups of training plants, wherein the data profiles from the two groups of plants have different identified phenotypes for a phenotype of interest, wherein the data profiles are obtained from the at least two groups of plants grown under the same conditions;
analyze or learn phenotype prediction from the data profiles using a supervised learning model;
obtain a data profile for a test plant; and
predict the phenotype of a phenotype of interest for the test plant.

19. The system of claim 18, wherein the phenotype of interest comprises disease resistance, drought tolerance, standability, yield, heat tolerance, cold tolerance, salinity tolerance, metal tolerance, herbicide tolerance, improved water use efficiency, nitrogen utilization, nitrogen fixation, pest resistance, or herbivore resistance.

20. The system of claim 18, wherein the data profile of the test plant and the data profiles of the at least two training groups of plants are the same type of data profiles, wherein the data profiles comprise genomic data profiles, transcriptomic data profiles, proteomic data profiles, metabolomic data profiles, spectral data profiles, or phenotypic data profiles.
